(19) United States
(12) Patent Application Publication
(10) Pub. No.: US 2015/0057530 A1
(43) Pub. Date: Feb. 26, 2015

(54) MEDICAL NEEDLE

(71) Applicant: KONINKLIJKE PHILIPS N.V., EINDHOVEN (NL)

(72) Inventors: Stephan Adriaan Roggeveen, Nuenen (NL); Gerhardus Wilhelmus Lucassen, Eindhoven (NL); Marjolein Van Der Voort, Valkenswaard (NL); Axel Winkel, Zapel-Hof (DE); Stephan Voss, Schwerin (DE); Jasper Klewer, Utrecht (NL); Bernardus Hendrikus Wilhelmus Hendriks, Eindhoven (NL); Waltherus Cornelis Jozef Bierhoff, Veldhoven (NL); Manfred Muller, Eindhoven (NL)

(21) Appl. No.: 14/387,959

(22) PCT Filed: Mar. 30, 2013
(86) PCT No.: PCT/IB2013/052573

§ 371 Date: Sep. 25, 2014

Related U.S. Application Data
(60) Provisional application No. 61/617,994, filed on Mar. 30, 2012.

(30) Foreign Application Priority Data
Jun. 15, 2012 (EP) 12172132.8

(51) Int. Cl. A61B 19/00; A61B 1/07; A61B 5/00; A61M 5/32

(52) U.S. Cl. CPC A61B 19/5244; A61M 5/3287; A61B 1/07; A61B 5/7264; A61B 5/4839; A61B 5/0084; A61B 19/201; A61B 2019/5255; A61M 2205/3306; A61M 2205/3303; A61M 2205/52; A61M 2205/3375; A61B 2019/202
USPC 600/424

(57) ABSTRACT

The present invention relates to a medical needle which comprises a needle (1) having at least one channel (21), at least one optical waveguide (22) and a syringe connector (20). The syringe connector (20) is in communication with the at least one channel (21) and permits further communication with an additional syringe (25), thereby permitting the correspondence of fluid between the syringe (25) and the tip of the needle (1). The optical waveguides (22) are arranged within the needle (1) in order to make optical measurements at the tip of the needle (1). The cross section of the distal end of the elongate tube (1) has a dividing line for each channel (21) which separates that channel (21) from the one or more optical waveguides (22).

### FIELD OF THE INVENTION

The invention relates to a medical needle which incorporates both a syringe for assisting in locating the position of the medical needle, and optical waveguides to perform optical measurements at the tip of the medical needle.

### BACKGROUND OF THE INVENTION

In the field of regional anesthesia and pain management it is common to perform nerve blocks i.e. to administer anesthetics near to nerves or inside the epidural space. In doing this it is important to be able to identify the Epidural Space (ES) and or nearby critical structures such as nerves and blood vessels. The gold standard for locating the ES is the Loss Of Resistance (LOR) method whereby the physician feels pressure loss on a saline- or air-filled syringe and connected tube to a needle entering the ES. When the needle tip enters the ES, the pressure on the syringe decreases with a consequent release of saline or air into the space which can be sensed by the physician on contact with the syringe.

One way to provide additional feedback on the needle tip location is to incorporate optical fibers in order to perform optical measurements at the tip of the needle. WO2011158227A2 discloses the combination of an optical spectroscopy technique with an expandable device located at the tip of a cannula to mechanically detect the transition between different tissues and cavities. WO2011158227A2 addresses the claimed limitations of the manual LOR technique which are i) “ . . . because of the elastic properties of the Ligamentum Flavum (LF), the elastic fibers are pushed by the needle and are stretched into the Epidural Space (ES)” [P3 L 10]”; ii) “ . . . Moreover, the resolution of the non-controlled advancement-increments of the needle tip is very limited and differs extensively from one physician to another” [P3 L 14]; and iii) “ . . . Another disadvantage of LORT is the relatively high risk of a false loss of resistance, taking place for instance inside the LF due to a small space between adjacent fibers”. In seeking to overcome such limitations WO2011158227A2 discloses to replace the manual LOR technique with an expandable device that gives feedback on the pressure exerted upon it at the tip of the needle. WO2011158227A2 further discloses the use of this device in conjunction with optical measurements at the tip of the needle.

The publication “Epidural needle with embedded optical fibers for spectroscopic differentiation of tissue: ex vivo feasibility study”, Desjardins et al. June 2011, Vol. 2, No. 6, Biomedical Optics Express 1452 also discloses the use of optical measurements in a medical needle in which a source optical waveguide and a detector optical waveguide are positioned either side of a channel and this has been found to deliver reasonable results.

### SUMMARY OF THE INVENTION

It is an object of the invention to provide a medical needle with improved positioning accuracy.

This object is achieved as claimed in claim 1 by the use of a medical needle in which the Loss Of Resistance (LOR) technique is executed at the same time as optical measurements. More reliable optical measurements are achieved by arranging the cross section of the needle at its distal end such that the cross section of the distal end of the elongate tube has a dividing line for each channel which is tangential to the cross section of that channel and transverse to the tube's longitudinal axis, and furthermore by arranging that the distal end of that channel lies on one side of said dividing line and the distal end of the one or more optical waveguides lie on the opposite side of said dividing line.

Furthermore it is found that when the optical measurements are combined simultaneously with the existing LOR technique in this way there is a surprising additional benefit that the “gold standard” LOR technique gives the physician confidence that the new technique is compatible with their training. In so doing the barrier to using a new technique such as optical spectroscopy, or a combination of new techniques such as disclosed in WO2011158227A2 is overcome.

According to a first aspect of the invention a medical needle is provided in the form of an elongate tube having an open distal end for insertion into the body and a proximal end. It will be appreciated that the distal end needs to be suitably shaped in order to penetrate the body, for example by making a bevel at the distal end. A syringe connector is provided in order to connect a syringe and thereby perform the Loss Of Resistance technique at the same time as making optical measurements at the distal end of the elongate tube. Furthermore it may be beneficial to use the same syringe that is employed in the LOR technique in the delivery of fluids into the body, for example anesthetic drugs. A channel is formed within the tube in order to facilitate the correspondence of fluid or air from the syringe to the open distal end of the elongate tube. At least one optical waveguide is provided in order to make optical measurements at the distal end of the elongate tube, the waveguides being used to guide light along the length of the elongate tube. The optical waveguide may be for example an optical fiber, a planar optical waveguide, or a light pipe.

The syringe connector is in communication with the channel close to the proximal end of the elongate tube. By sensing the pressure on a syringe connected to the syringe connector the medical practitioner senses the position of the needle with respect to the Epidural Space. Optionally the total cross sectional area of the channel is no less than 5% of the outer cross sectional area of the elongate tube in order that pressure at the open distal end of the elongate tube can be adequately sensed through use of the syringe. The syringe connector may be in communication with the channel at the extreme proximal end of the elongate tube, or alternatively the communication may be made through for example the wall of the elongate tube close to the proximal end. Examples of suitable syringe connectors include a Luer connector or a push-fit tubing connector, both examples being found in the medical field. A push-fit tubing connector permits the connection of the needle to a syringe via push-fit tubing, and is sometimes a feature of LOR techniques. The push fit tubing allows the syringe to be located away from the elongate tube and has the benefit firstly of improving physician workflow and secondly of preventing the risk that the needle position is disturbed when pressure is applied to the syringe.

At least one optical waveguide is arranged within the elongate tube. In order to make optical measurements at the distal end of the elongate tube, at least one optical waveguide communicates with an optical source at the proximal end of the elongate tube, and at least one optical waveguide communicates with an optical detector at the proximal end of the elongate tube. A suitable optical source provides optical radiation within the range extending from 0.1 μm to 100 μm, optionally in the region from 0.3 μm to 2.5 μm. A suitable optical detector is one that is arranged to measure one or more optical properties of the radiation and to generate a response, for example from the intensity, wavelength or phase. Suitable means for facilitating the optical communication with an optical source and an optical detector include an optical fiber, a planar optical waveguide, or a light pipe. In some examples of the invention the one or more optical waveguides facilitating this communication are the same one or more optical waveguides that are arranged within the tube, but this is not necessarily always the case. Optical radiation from an optical source is guided by the at least one optical waveguide to the distal end of the elongate tube where it irradiates tissue in the vicinity of the distal end. The radiation is subsequently reflected and scattered by this tissue. Subsequently a portion of this radiation is collected by the distal end of the at least one optical waveguide that communicates with an optical detector and the detector generates a response to this portion. Optionally the detector is further arranged to generate a response to the optical source radiation in order to compare it with the response from the scattered and reflected radiation.

During execution of the LOR technique it has been found that fluid or air emitted at the distal end of the elongate tube by the channel in communication with the syringe may interfere with the optical measurements at the distal end of the elongate tube. According to a first aspect of the invention the cross section of the distal end of the elongate tube has a dividing line for each channel which is tangential to the cross section of that channel and transverse to the tube's longitudinal axis. Furthermore, the distal end of that channel is arranged to lie on one side of said dividing line and the distal end of the one or more optical waveguides are arranged to lie on the opposite side of said dividing line. By separating the one or more optical waveguides from the one or more channels in this way, fluid or air emitted by the one or more channels during the LOR technique is emitted away from the one or more optical waveguides. This substantially prevents fluid or air from interrupting the optical path between the optical source and the optical detector at the distal end of the elongate tube. By arranging the one or more channels and one or more optical waveguides in this way, superior results to those obtained in “Epidural needle with embedded optical fibers for spectroscopic differentiation of tissue: ex vivo feasibility study”, Desjardins et al. June 2011, Vol. 2, No. 6, Biomedical Optics Express 1452 have been achieved. This is due to the prevention of the fluid or air emitted at the distal end of the one or more channels from interrupting the field of view of the optical waveguides. An exemplary extreme situation that is precluded by this aspect of the invention is where a channel is located between an optical waveguide in communication with the optical source and an optical waveguide in communication with the optical detector. In this extreme situation, fluid or air emitted by the channel during the LOR technique has been found to interfere with the optical measurements and consequently this situation is avoided.

According to a second aspect of the invention the distal end of the at least one optical waveguide is fixed with respect to the long axis of the elongate tube. This prevents the one or more optical waveguides from moving with respect to the elongate tube when it is inserted into the body. If the optical waveguides were to move during insertion the resulting change in irradiation profile or the change in collected radiation could be misinterpreted. Furthermore, in the event that any fluid or air does interrupt this optical path when the distal end of the at least one optical waveguide is thus fixed, the interference with the optical measurements is minimized since the fluid or air has the same effect on the optical measurements whenever such fluid or air is present. By so fixing the distal end of the at least one optical waveguide with respect to the long axis of the elongate tube, even more reliable optical measurements can be made.

According to a third aspect of the invention, the elongate tube has a single bore into which the one or more optical waveguides are inserted. This simplifies the manufacture of the elongate tube which is easier for tubes having a single bore than for tubes with multiple bores. According to this aspect of the invention the channel that is in communication with the syringe connector is formed within the same bore that has one or more optical waveguides inserted in it.

According to a fourth aspect of the invention the elongate tube has two or more bores which are mutually isolated along the length of the tube. Furthermore, the one or more optical waveguides are inserted into one or more of these bores. Broadly, a bore may be designated as a channel in communication with the syringe connector, or designated as having one or more optical waveguides inserted therein. Alternatively a bore may have a channel formed therein, as well as have one or more optical waveguides inserted therein. In one example of the invention there are three bores in which one bore is designated for use as a channel and is in communication with the syringe connector, the two further bores each having a single optical waveguide inserted therein. In another example there are four bores in which two bores are dedicated for use as channels in communication with the syringe connector, and the two further bores each have a single optical waveguide inserted therein.

According to the fifth aspect of the invention the stylet insert is further defined to have at least one lumen. At least one optical waveguide is arranged within the at least one lumen of the stylet insert, and the stylet insert is further inserted into the single bore of the elongate tube. The lumens and the stylet insert act to arrange the one or more channels with respect to the dividing line in accordance with the first aspect of the invention. Furthermore, by grouping the optical waveguides together, the stylet insert facilitates the easier insertion of the optical waveguides into the bore in the elongate tube. In this aspect of the invention having a single bore the channel is formed within the same bore that has the stylet insert inserted into it.

According to a sixth aspect of the invention it is arranged that at least a portion of the outer cross section of the stylet insert fits to the inner cross section of the bore into which it is inserted. Furthermore, it is arranged that for this portion the outer surface of the stylet insert is in intimate contact with the inner cross section of the bore into which it is inserted. In so doing the stylet insert and consequently the one or more optical waveguides inserted into its one or more lumens are fixed with respect to the long axis of the elongate tube. The waveguides are thereby rendered immobile with respect to the elongate tube, in particular when the distal end of the tube is inserted into the body.

According to a seventh aspect of the invention the distal end of the elongate tube has a bevel and the distal end of the stylet insert has a bevel with substantially the same bevel angle. Furthermore, the stylet insert is arranged within the elongate tube such that the bevel of the stylet insert and the bevel of the elongate tube are substantially coincident. A bevel is a useful profile to apply to the distal end of the elongate tube in order to make it easier to penetrate the body. Furthermore, by arranging that the stylet insert has substantially the same bevel angle, and that the bevels are substantially coincident, the distal end of the elongate tube the stylet insert is prevented from interfering with the penetration mechanics of the elongate tube as it penetrates the body.

According to an eight aspect of the invention there is at least one optical waveguide in communication with an optical source, namely a source optical waveguide, and at least one optical waveguide in communication with an optical detector, namely a detector optical waveguide. Further, the at least one source optical waveguide is separate to the at least one detector optical waveguide. By thus separating the functionality of the optical waveguides a simpler communication with the optical source and optical detector is facilitated.

According to a ninth aspect of the invention the end face of the beveled distal end of the elongate tube has a dividing line. Furthermore, the distal end of the at least one source optical waveguide is arranged to lie on a first side of said dividing line and the distal end of the at least one detector optical waveguide is arranged to lie on a second side of said dividing line. Optionally the dividing line is parallel to the short axis of the bevel. By so separating the optical waveguides the one or more source optical waveguides have a large separation at the distal end from the one or more detector optical waveguides. The depth into the tissue in contact with the distal end that is sensed by this optical waveguide arrangement is dependent upon the separation between the source optical waveguides and the detector optical waveguides at the distal end; larger separations giving rise to deeper sensing. By arranging the optical waveguides in this way, deeper sensing into the tissue is facilitated. This arrangement is particularly advantageous in for example narrow gauge needles in which deeper sensing is desired.

According to a tenth aspect of the invention the at least one optical waveguide comprises at least one optical fiber. Optical fibers have the advantage of ease of manufacture and are suited to the guidance of optical radiation, which is guided by refractive index differences between the core and the cladding. An optical fiber suited to this purpose may have for example a glass core or a polymer core. Optionally the at least one optical fiber is further coated at its distal end in order to protect the fiber or furthermore to assist in the coupling of light, for example by applying an antireflection coating. Example coatings for these purposes include magnesium fluoride, diamond-like carbon, and fluoropolymers. Optionally it is arranged that the core of the at least one optical fiber at its distal end defines a plane which is substantially normal to the long axis of the optical fiber. This assists in reducing the interface reflectance within the optical fiber that might otherwise prevent the efficient coupling of light out of the optical fiber. Likewise, this arrangement assists in improving the coupling of light into the optical fiber. Cleaving is a suitable technique for producing an optical fiber in which the core at its distal end defines a plane which is substantially normal to the long axis of the optical fiber. In order to cleave an optical fiber, the fiber is typically placed under tension, scribed with a diamond or carbide blade perpendicular to the axis, and then the fiber is pulled apart to produce a clean break. Alternatively, polishing may be used to produce such a termination to the optical fiber. Optionally the plane defined by the core at the distal end of the at least one optical fiber is a few degrees away from being normal to the long axis of the optical fiber. For optical radiation emitted by an optical fiber, as the angle of this plane is decreased from the normal at 90 degrees towards zero the net interface reflectance increases until total internal reflection occurs, at which point no light leaves the end of the optical fiber. However an optical fiber having a core which defines a plane that is normal to the long axis of the optical fiber risks sending such interface reflections straight back into the optical source where the reflections may interfere with the optical source or further cause spurious optical effects when detected. By arranging that the core of the optical fiber at its distal end defines a plane that is a few degrees away from being normal to the long axis of the optical fiber, typically 8 degrees away from the normal, it is arranged that such interface reflections are directed toward the cladding of the optical fiber where they are inefficiently guided back toward the source. Thus it may be desirable to so shape the distal end of the at least one optical fiber. Polishing is a suitable technique to shape the end of the optical fiber at a non-normal angle to the long axis of the optical fiber. When using non-normal terminations to the optical fibers the optical fiber cladding and optical fiber buffer materials may optionally be chosen in order that they do not significantly influence the optical spectrum within the range detected by the optical detector. Optical radiation which passes through the cladding and buffer layers close to the end face of the optical fiber as a consequence of its reflectance at the end face may irradiate and be scattered and reflected by tissue in the vicinity of the end face and subsequently be guided to the optical detector. By so selecting the optical fiber cladding and optical fiber buffer materials, stray radiation irradiating the tissue via the cladding and buffer layers does not affect the spectrum of the detected signal.

According to an eleventh aspect of the invention at least one optical connector at the proximal end of the elongate tube is further provided. Furthermore, the at least one optical waveguide is in communication with an optical source by means of an optical connector, and the at least one optical waveguide is in communication with an optical detector by means of an optical connector. In so doing the one or more optical connectors facilitate the temporary attachment of the optical source and optical detector to the waveguides in the elongate tube during use, allowing for the later disposal of the elongate tube with the waveguides contained therein. The optical connector optionally provides both optical communication as well as mechanical registration in order to prevent disturbance of the optical communication during relative movement between the elongate tube and the optical source and optical detector. Examples of optical connectors that are suited to this aspect and which provide both optical and mechanical registration include but are not limited to ST, SC, FC, SMA, FDDI, Mini-BNC, MT-RJ style connectors. In one example of the invention there are two optical waveguides inserted into the tube; a first optical waveguide in communication with an optical source and a second optical waveguide in communication with an optical detector. In this example the communication with the optical source is made by means of an optic fiber, and likewise the communication with the optical detector is made by means of a separate optical fiber. In this example the communication between the first optical fiber and the corresponding optical fiber in communication with the optical source is made by means of an SMA optical connector. Likewise the communication between the second optical fiber and the corresponding optical fiber in communication with the optical detector is made by means of a separate SMA optical connector.

According to a twelfth aspect of the invention at least one mechanical fastening at the proximal end of the elongate tube is further provided. According to this aspect the at least one optical waveguide in communication with an optical source at the proximal end of the elongate tube is fixed with respect to the elongate tube by means of a mechanical fastening, and the at least one optical waveguide in communication with an optical detector at the proximal end of the elongate tube is fixed with respect to the elongate tube by means of a mechanical fastening. In so doing the at least one mechanical fastening facilitates the temporary insertion of the one or more waveguides in communication with the optical source, and the one or more waveguides in communication with the optical detector, into the elongate tube during use, allowing for the later disposal of the elongate tube. In one example of this aspect there may be two optical waveguides, one in communication with an optical source and one in communication with an optical detector. Optionally each optical waveguide is continuous in the sense that when inserted into the needle, one end of the optical waveguide is at the distal tip of the needle and the other end of the optical waveguide is located at the respective source or optical detector. A suitable waveguide for this purpose is for example an optical fiber. The mechanical fastening serves the purpose of temporarily registering the position of the optical waveguide with respect to the needle during use, and permits its removal and cleaning prior to a subsequent use, whilst the needle is discarded. Examples of mechanical fastenings that are suited to this aspect of providing temporary registration include but are not limited to screw-threaded fastenings and snap fastenings.

According to a thirteenth aspect of the invention at least one optical waveguide is formed within the elongate tube's inner surface. According to this aspect, optical radiation propagates to and from the distal end of the elongate tube by means of reflections from the tube's inner surface. The region in which the optical radiation propagates is optionally substantially filled with one of air, fluid, vacuum or a gas to assist in the guidance of light. One advantage resulting from this option is the reduced cost of the components in the needle. Another advantage is the reduction in cleaning requirements for this optional implementation of the one or more optical waveguides. Optionally the inner surfaces of the elongate tube acting as waveguides are further coated with materials to improve the optical waveguiding properties, for example by coating them with a metallic or polymer layer. According to this aspect, radiation is launched into the waveguide formed within the elongate tube's inner surface by for example an optical fiber in communication with an optical source, wherein the optical fiber does not extend, or extends only partially into the proximal end of the elongate tube in order to assure mechanical registration between the optical fiber and the inner surface of the waveguide. Likewise, optical radiation collected at the distal end of the waveguide formed within the inner surface of the elongate tube is guided by reflections from the inner surface of the elongate tube to for example an optical fiber an optical fiber that is in communication with an optical detector, wherein the optical fiber does not extend, or extends only partially into the proximal end of the elongate tube. In one example an optical waveguide is formed within the same bore as that in communication with the syringe connector, and in this example the optical guidance medium is the same fluid or air that is used in the syringe in the Loss Of Resistance technique. In another example an optical waveguide is formed within a separate bore to that in communication with the syringe connector.

According to a fourteenth aspect of the invention a look-up table comprising the optical properties of human tissue at optical wavelengths is further provided. The optical detector is further arranged for the generation of a response to radiation collected at the distal end of the elongate tube. Further, the type of tissue in contact with the distal end of the elongate tube is determined from the optical response and the look-up table. The optical properties of tissue stored in the look-up table include for example the reflectance values of different tissues at different wavelengths. In so doing the optical measurements are used to discriminate between different types of tissue in contact with the distal end of the elongate tube.

According to a fifteenth aspect of the invention a needle positioning arrangement is disclosed. This comprises the medical needle in claim 1 being further provided with a syringe connected to the syringe connector, wherein the syringe is in communication with an Acoustic Pressure Assist Device (APAD). The use of the APAD device with the standard Loss of Resistance technique per se is known from for example Lechner T. M. J., van Wijk M. G. F., Maas A. J. J. et al. “The use of a sound-enabled device to measure pressure during insertion of an epidural catheter in women in labour”. Anaesthesia, 2011; 66: 568-573. The APAD operates by applying pressure to the syringe and gives continuous acoustic feedback to the physician relating to the pressure exerted by the syringe at the distal end of the elongate tube. As the needle is inserted into the body the changes in pressure at the tip of the needle are translated into changes in pitch that are heard by the physician. The epidural space is identified by the sudden change in pitch of the acoustic signal as the Loss Of Resistance occurs. The syringe is typically connected to the syringe connector by means of an extension tube to permit the APAD device to be located away from the patient. The APAD thus automates the pressure feedback element to the LOR technique. By using the medical needle in combination with the APAD the benefit achieved is that the use of the medical needle is further simplified. Thus once the practitioner has the confidence from the manual LOR technique that the new optical measurement technique works, the use of the medical needle with the APAD provides the physician with simpler, improved positioning of the medical needle within the epidural space.

### BRIEF DESCRIPTION OF THE FIGURES

FIG. 1 shows schematically the anatomical structures of the spinal column in which a needle penetrates these structures in order to deliver anesthetic reagents to the epidural space during epidural anesthesia.

FIG. 2 shows schematically the relationship between some elements of the invention, an additional syringe, an additional optical source and an additional optical detector

FIG. 3 shows schematically the tip of a needle in three views as an example of a first embodiment of the invention having a stylet insert FIG. 4 shows schematically the tip of a needle in top view in exemplary arrangements of the first embodiment of the invention

FIG. 5 shows schematically the tip of a needle in three views as an example of a second embodiment of the invention

FIG. 6 shows schematically the tip of a needle in top view in exemplary arrangements of the second embodiment of the invention

FIG. 7 shows graphically the absorption of different biological chromophores as a function of optical wavelength

FIG. 8 shows schematically an example arrangement of the invention being further provided with an optical connector at the proximal end of a needle

FIG. 9 shows schematically an example arrangement of the invention being further provided with a mechanical fastening at the proximal end of a needle

### DETAILED DESCRIPTION OF THE INVENTION

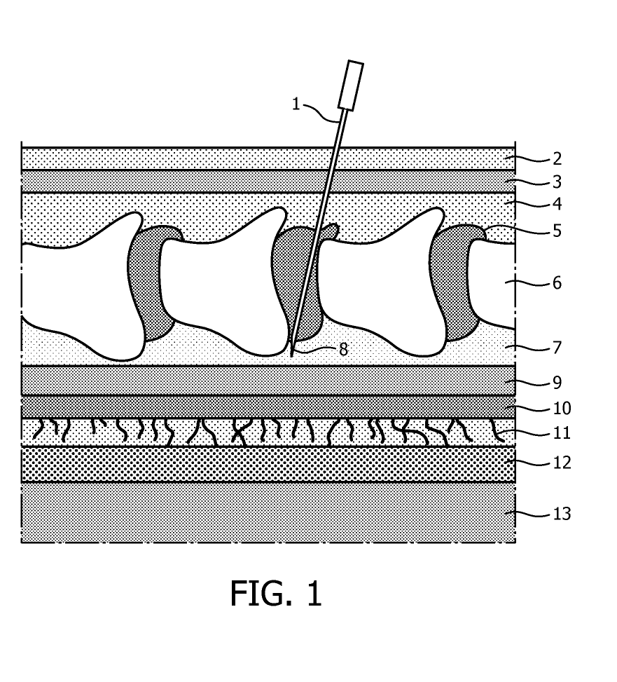

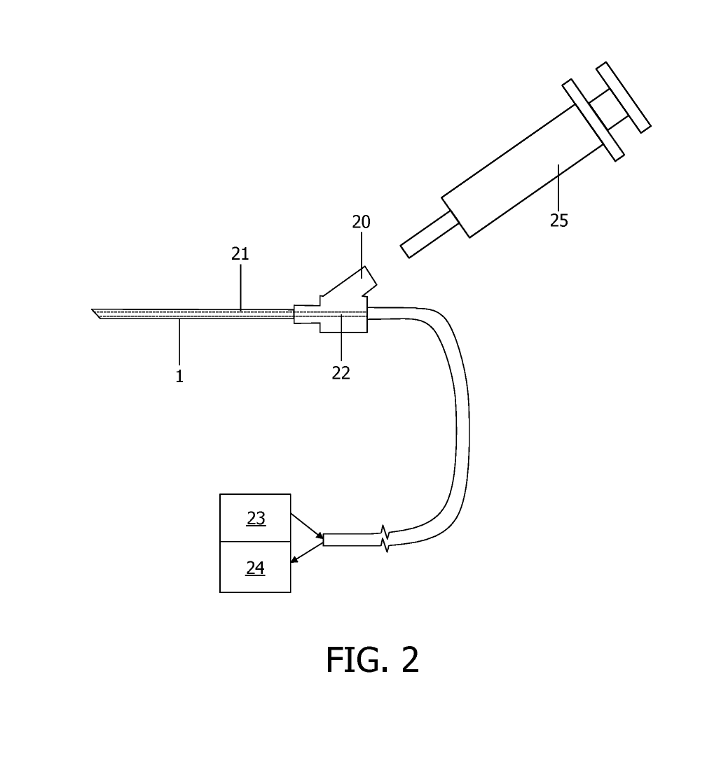

In order to provide a medical needle with improved positioning accuracy, various embodiments of a medical needle are now described in an exemplary application of epidural anesthesia. FIG. 1 shows schematically the anatomical structures of the spinal column in which a needle 1 penetrates these structures in order to deliver anesthetic reagents to the epidural space 7 during epidural anesthesia. In this example it is desired to place the needle tip 8 within the Epidural Space 7 and to subsequently inject anesthetic reagents into this Epidural Space. The needle must penetrate the skin 2, Subcutaneous fat 3, Supraspinous ligament 4, Interspinous ligament 5 that separates Vertebral bones 6 in order to reach the Epidural Space 7. Once the dense Interspinous ligament 5 has been pierced the anesthetist advancing the needle feels a sudden drop in pressure at the needle tip, or a Loss Of Resistance as the needle advances into the Epidural Space 7. If the needle penetrates too far it risks damage to the underlying Dura Mater 9, Arachnoid 10, Pia Mater 12 and Spinal cord 13. If the needle were advanced into the subarachnoid space 11 and anesthetic reagents were released there, spinal anesthesia would be performed whose effects are different to that of epidural anesthesia. It is therefore desired in this exemplary application to improve the positioning accuracy of such an epidural needle in the Epidural Space 7 within the spinal column. It is however noted that the present invention can also be applied to another medical probe which can be, for instance, any slender, surgical instrument with a tip, used to explore other body tissue such as a wound or body cavity.

FIG. 2 shows schematically the relationship between some elements of the invention, an additional syringe 25, an additional optical source 23 and an additional optical detector 24. In FIG. 2, needle 1 has a syringe connector 20 and a channel 21 which permits the correspondence of fluid from the syringe connector to the open distal end of the needle. One or more optical waveguides 22 are inserted into the needle 1 in order to facilitate optical measurements of tissue in the vicinity of the needle tip. By distributing the optical waveguides in the needle so as to provide a channel it becomes possible to perform pressure measurements simultaneously with optical measurements. One or more optical waveguides 22 communicate with an additional optical source 23, and one or more optical waveguides communicate with an additional optical detector 24 at the proximal end of the needle. During use, an additional syringe 25 is mated with syringe connector 20 in order to apply pressure at the distal end of the needle 1 to provide compatibility with the LOR technique as the needle is inserted into the body. It is noted that during use the mating between the syringe 25 and the syringe connector 20 may be facilitated by means of a tube or other fluidic connector in order to locate the syringe further away from the needle in order to improve workflow for the physician.

The solution in FIG. 2 can be further improved by arranging the cross section at the distal end of the elongate tube such that the cross section of the distal end of the elongate tube has a dividing line for each channel which is tangential to the cross section of that channel and transverse to the tube's longitudinal axis, and furthermore by arranging that the distal end of that channel lies on one side of said dividing line and the distal end of the one or more optical waveguides lie on the opposite side of said dividing line. By separating the one or more optical waveguides from each channel in this way, fluid or air emitted by the one or more channels during the LOR technique is emitted away from the one or more optical waveguides. This substantially prevents fluid or air from interrupting the optical path between the optical source and the optical detector at the distal end of the elongate tube. By so arranging the one or more optical waveguides and one or more channels at the distal end of the elongate tube, more reliable optical measurements can be made during the simultaneous execution of the LOR technique.

The solution in FIG. 2 can be even further improved by fixing the distal end of the at least one optical waveguide with respect to the long axis of the elongate tube. The fixation firstly prevents the one or more optical waveguides from moving with respect to the elongate tube when it is inserted into the body. If the optical waveguides were to move during insertion, changes in the irradiation profile or changes in the collected radiation could be misinterpreted. Secondly, the fixation ensures that if any fluid or air does interrupt the optical path between the optical source and optical detector its effect on the optical measurement is the same whenever such fluid or air is present and can thus be corrected-for. Thus, by fixing the distal end of the at least one optical waveguides with respect to the long axis of the elongate tube, more reliable optical measurements can be made.

The following embodiments relate to the example of a medical needle to which the invention can be applied.

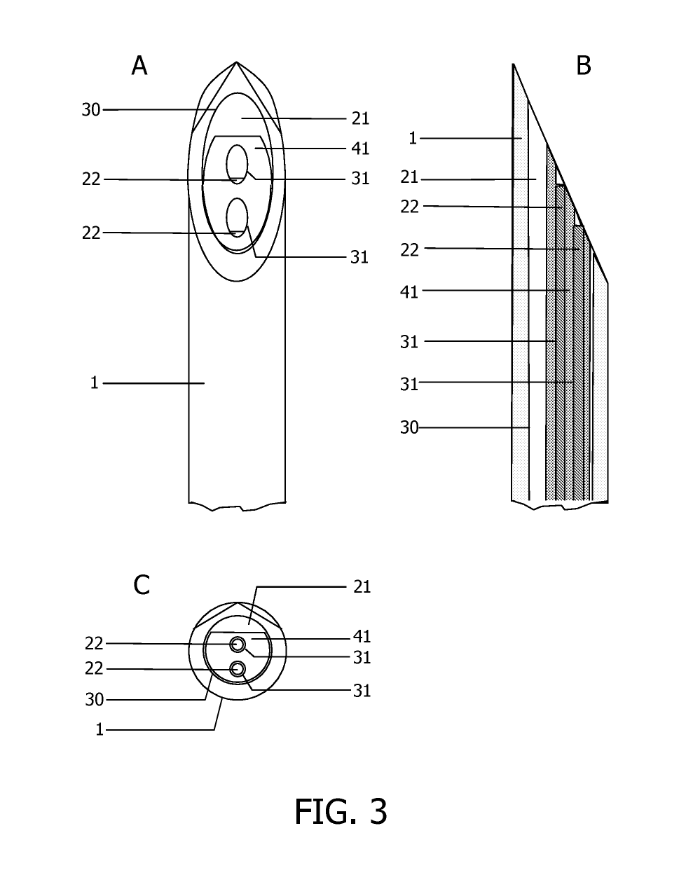

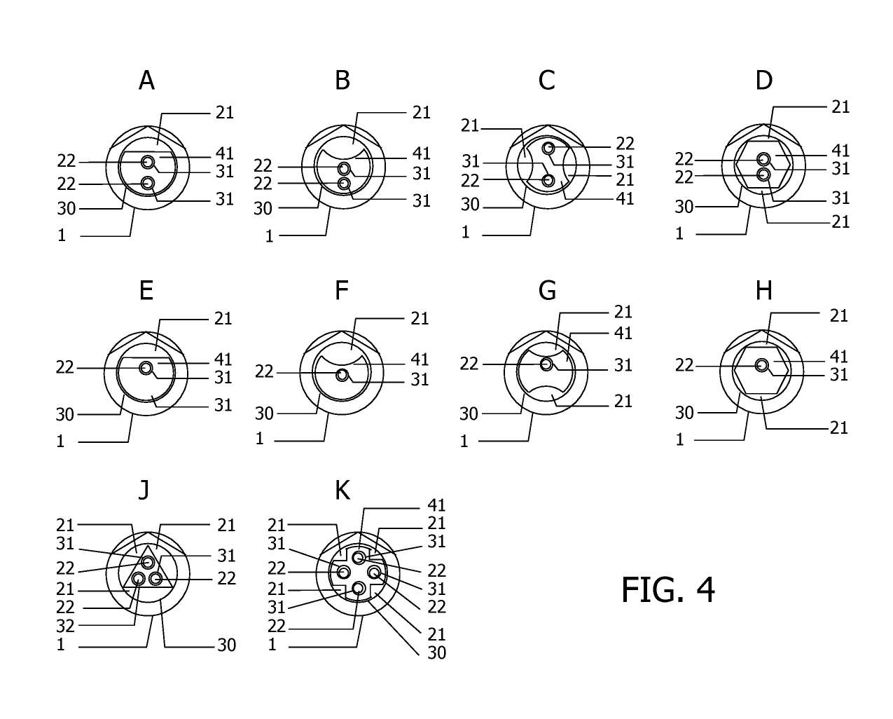

FIG. 3 shows schematically the tip of a needle in three views as an example of a first embodiment of the invention having a stylet insert. The stylet insert arranges the one or more optical waveguides at the distal end of the needle such that in cross section each channel has a dividing line which is tangential to the cross section of that channel and transverse to the tube's longitudinal axis and furthermore by arranging that the distal end of that channel lies on one side of said dividing line and the distal end of the one or more optical waveguides lie on the opposite side of said dividing line. By separating the one or more optical waveguides from each channel in this way, fluid or air emitted by the one or more channels during the LOR technique is emitted away from the one or more optical waveguides, thereby improving the reliability of the optical measurements. In FIG. 3, a needle with a single bore 30 into which one or more optical waveguides 22 are inserted is shown in front A, side B, and top C projections. One example of a suitable needle to which this embodiment can be applied is an 18 Gauge epidural cannula, although the invention is not limited to this example. The one or more optical waveguides are arranged according to the dividing line by inserting them in one or more lumens 31 of a stylet insert 41, and inserting the stylet insert into the bore 30 of the needle 1. The construction of a stylet insert per se is known in the field of medical devices and is typically constructed from polymers such as Polyethylene Terephthalate (PET), Polyethylene (PE), High Density Polyethylene (HDPE), Polyvinyl Chloride (PVC), Polypropylene (PP), Polystyrene (PS), and Polycarbonate (PC). The cross section of stylet insert 41 is shaped such that it does not completely fill the bore 30 into which it is inserted, thereby leaving a channel 21 which is separated from the optical waveguides according to the dividing line. The stylet insert embodiment may optionally be further improved by arranging that the distal end of the at least one optical waveguide is fixed with respect to the long axis of the needle. In the absence of this optional arrangement the distal ends of the optical waveguides are arranged according to the dividing line in the first aspect of the invention, but the distal ends would be capable of moving with respect to the long axis of the needle, risking that movement of the one or more waveguides during insertion into the body interferes with the optical measurements. This optional arrangement is also shown in FIG. 3 in which the distal end of the at least one optical waveguide fixed with respect to the long axis of the tube by arranging that at least a portion of the outer cross section of the stylet insert 41 fits to the inner cross section of the bore 30 into which it is inserted, such that for this portion the outer surface of the stylet insert is in intimate contact with the inner cross section of the bore into which it is inserted. This is shown by way of the example in FIG. 3C in which part of the cross section of the stylet insert 41 is circular, and this fits to the circular inner cross section of the needle bore 30. In FIG. 3 there are two optical waveguides but in other examples there may be one or more optical waveguides. Further examples of this embodiment having a stylet insert are shown in FIG. 4, which shows schematically the tip of a needle in top view in exemplary arrangements of the first embodiment of the invention. In the examples A to K shown in FIG. 4, and in FIG. 3, the needle has a single bore, and the stylet insert is arranged to provide a channel 21 by leaving part of the cross section of the bore free from the stylet insert. Other shapes of stylet insert cross section which achieve the desired function of arranging the cross section at the distal end of the elongate tube such that each channel lies on one side of a dividing line that separates it from the one or more optical waveguides engaged in optical measurements are within the scope of the invention. By separating the one or more optical waveguides from each channel in this way, fluid or air emitted by the one or more channels during the LOR technique is emitted away from the one or more optical waveguides. are within the scope of this embodiment. The use of more than one channel assists in more evenly distributing the fluid when this is injected into the Epidural Space during the LOR technique. Likewise it assists in ensuring that the pressure sensed through the syringe is an average of that applied at the tip of the needle. Optionally, as shown in FIG. 3 and FIG. 4, the distal end of the at least one optical waveguide is further fixed with respect to the long axis of the elongate tube. Optionally, according to this embodiment there are two optical waveguides and the stylet insert is of the shape shown in FIG. 4A, having a flat section cut away from an otherwise circular shape.

A dividing line referred-to throughout the application is defined in more detail below with particular reference to the first embodiment illustrated in FIG. 3 and in FIG. 4. A dividing line is a property of each channel; thus each channel may have a different dividing line. A dividing line is a straight line which passes through a point on the channel boundary, the line being tangential to the cross section of that channel and transverse to the tube's longitudinal axis. A dividing line defines the position of any channel with respect to the two or more optical waveguides. Its purpose is to ensure that no portion of a channel exists within the region encompassed by a virtual rubber band stretched around the cross sectional perimeter of the two or more optical waveguides. Interference with optical measurements is reduced by arranging that the distal end of that channel lies on one side of said dividing line and the distal end of the two or more optical waveguides lie on the opposite side of said dividing line. If a portion of a channel were located within the region defined by the rubber band above it would interrupt the optical path comprising light delivered and light received by the optical waveguides, thereby degrading the quality of the optical measurements.

With further reference to the first embodiment comprising a stylet insert, the formation of a channel is now described in more detail with reference to examples in FIG. 3 and in FIG. 4. In general a channel may be formed by arranging that the distal end of the cross section of a stylet insert is shaped such that it does not completely fill the bore into which it is inserted; thus a channel is formed by leaving part of the cross section of the bore free from the stylet insert. In this way a channel is formed by an outer surface of the stylet insert and an inner surface of the bore. FIG. 3A illustrates a single channel formed in this way, although a plurality of channels may be formed in the same way. By forming channels in this way a simpler construction of the stylet insert is achieved because there is no need to form a separate lumen within the stylet insert to act as a channel. Furthermore the quality of sterilisation of the stylet insert is improved because the stylet insert has only external surfaces that require sterilisation. The needle tube component may be sterilized using existing needle sterilisation techniques.

With exemplary reference to FIG. 3A, a channel 21 is formed by arranging that the distal end of the cross section of the stylet insert 41 is shaped such that it does not completely fill the bore 30 into which it is inserted and the at least one channel 21 is formed by leaving part of the cross section of the bore 30 free from the stylet insert 41; thus such that the channel is formed by an outer surface of the stylet insert and an inner surface of the bore 30. Channels are formed in the same way in FIG. 4. With continued reference to FIG. 3, channel 21 has a dividing line which meets the condition: the cross section of the distal end of the elongate tube 1 has a straight dividing line for each channel wherein the dividing line passes through a point on the channel boundary and is tangential to the cross section of that channel and transverse to the tube's longitudinal axis wherein the distal end of that channel is arranged to lie on one side of said dividing line and the distal ends of the two or more optical waveguides are arranged to lie on the opposite side of said dividing line. Such a dividing line can be constructed in exemplary FIG. 3C by drawing a line parallel to and passing through a point on the flat edge of the stylet insert 41 in the cross sectional illustration in FIG. 3C. Dividing lines conforming to this condition can likewise be constructed for each channel in the examples in FIG. 4A-FIG. 4D and FIG. 4J, FIG. 4K.

By way of another example, cross sectional illustration FIG. 4K comprises four channels 21 wherein a dividing line meeting the same above condition can be constructed for each of the four channels. For the channel in the top left corner a first dividing line can be constructed that passes through a point on the right-angled corner of the channel boundary, the line running in a South West to North East direction for which all four optical waveguides 22 lie on one side, thus the lower side of the dividing line and the distal end of the channel lies on the other, thus the upper side of the dividing line. Likewise a dividing line that is parallel to the first dividing line may be constructed which passes through a point on the right-angled corner of the channel boundary of the bottom right channel for which all four optical waveguides 22 lie on one side, thus the upper side of the dividing line and the distal end of the channel lies on the other, thus the lower side of the dividing line. Orthogonal lines meeting the same dividing line condition can likewise be constructed for the top right and bottom left channels in FIG. 4K.

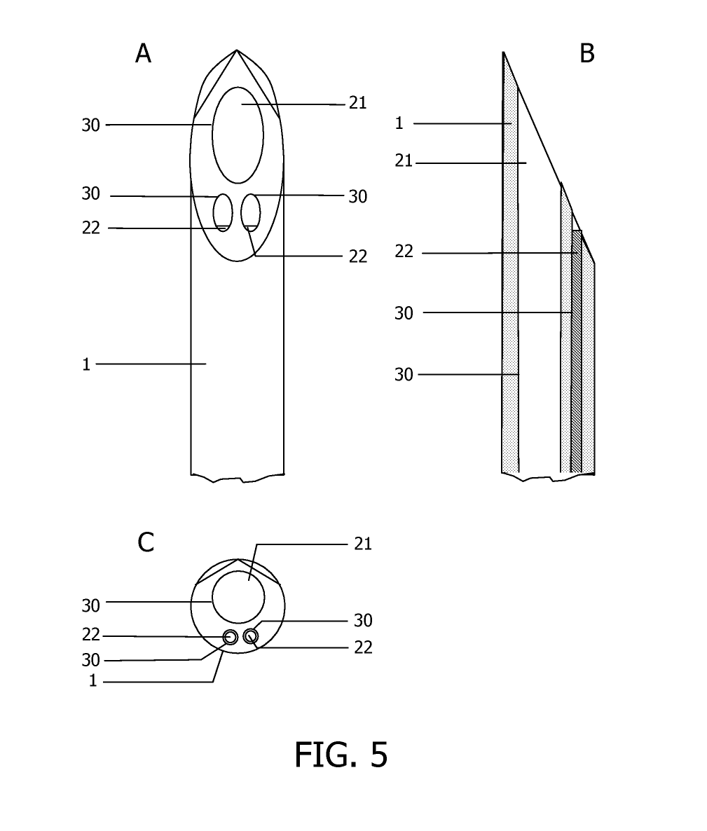

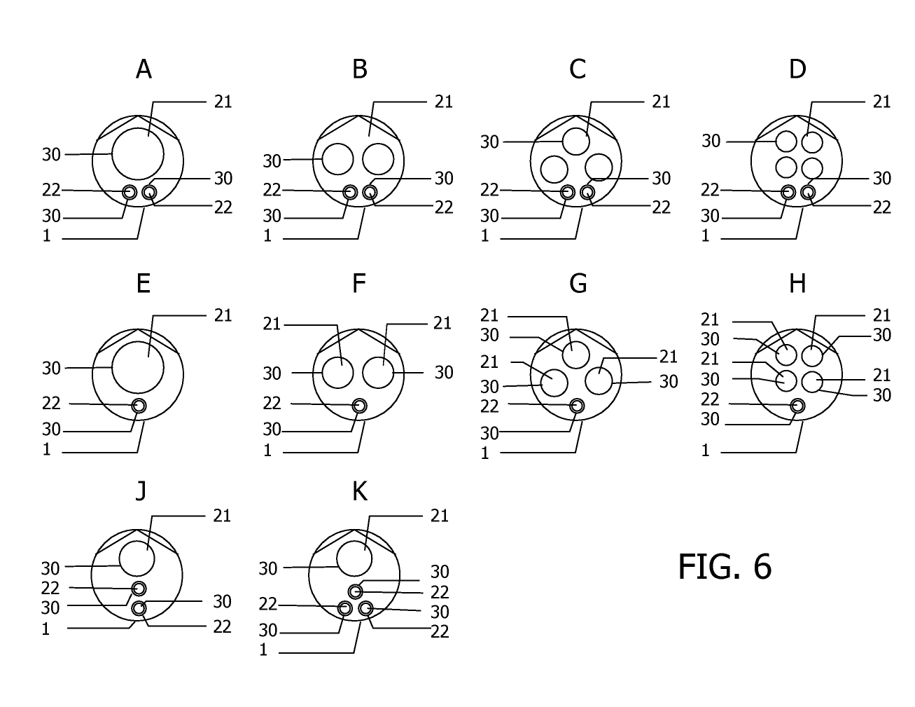

According to a second embodiment of the invention the needle has two or more bores which are mutually isolated along the length of the elongate tube, and the one or more optical waveguides are inserted into one or more of these bores. By inserting the one or more optical waveguides into the one or more bores the distal ends of the one or more optical waveguides are arranged according to the dividing line of the first aspect of the invention, thereby improving the reliability of the optical measurements. FIG. 5 shows schematically the tip of a needle in three views as an example of a second embodiment of the invention. In FIG. 5, front A, side B, and top C projections are shown in which there are three bores 30, two of which each have an optical waveguide 22 inserted therein, the third bore being dedicated to use as a channel 21. Further examples are shown in FIG. 6 which shows schematically the tip of a needle in top view in exemplary arrangements of the second embodiment of the invention. Further examples of the second embodiment shown in FIG. 6 have two optical waveguides in A to D, one optical waveguide in E to H and further arrangements of waveguides within the bores in J and K. It may be beneficial to use more than one bore as a channel in order to maintain the structural properties of the needle, or furthermore to insert additional sensors into these bores during use of the medical needle. In cases where there is more than one channel, there is a dividing line according to the first aspect of the invention for each channel. Thus for example in FIG. 6D in which there are four channels and two optical waveguides, each of the four channels has a separate dividing line which in cross section is tangential to that specific channel and which can be placed such that the channel lies on one side of the dividing line and the one or more, in this example, two, optical waveguides all lie on the other side of the dividing line. Optionally the one or more optical waveguides are further fixed with respect to the long axis of the needle 1 in order to even further improve the reliability of the optical measurements, for example by securing each waveguide within its respective bore using epoxy resin.

According to the first and the second embodiments there is at least one optical waveguide in communication with an optical source 23 at the proximal end of the elongate tube, and at least one optical waveguide in communication with an optical detector 24 at the proximal end of the elongate tube. This is shown schematically in FIG. 2. The optical source 23 generates optical radiation in the region from 0.1 μm to 100 μm, optionally in the region from 0.3 μm to 2.5 μm, which is guided by a first optical waveguide 22 to the distal end of the needle where it irradiates tissue in the vicinity of the tip. Optical radiation is then scattered and reflected by this tissue, the optical properties of said tissue conferring upon the reflected and scattered radiation some specific optical characteristic. A portion of the reflected and scattered light is collected at the distal end of a second optical waveguide that guides the light back to the optical detector 24. A suitable optical source may be for example a halogen lamp, LED, fluorescent lamp, laser, UV tube or thermal source, or a selection of these sources to provide the desired spectral coverage. The optical source may further be filtered using for example a bandpass, a short pass or a long pass filter in order to limit the optical spectrum that is subsequently guided to the distal end of the optical waveguide. The optical detector 23 is configured to measure for example the intensity, wavelength and phase of the optical radiation collected at the distal end of the waveguide. Furthermore, the optical radiation falling on the optical detector may be filtered using for example a bandpass, a short pass or a long pass filter in order to limit the optical spectrum that is subsequently detected. The described combination of optical source and optical detector is optionally arranged to form what is better known as a spectrometer, a spectrophotometer, diffuse reflectance spectroscopy system, fluorescence spectroscopy system, an optical coherence spectroscopy system, a Raman spectroscopy system, coherent Raman spectroscopy system, optical spectroscopic or microscopic imaging modalities or a wavelength-selective power meter. By so measuring the optical radiation the optical characteristics of the different tissue in the vicinity of the tip of the needle can be used to distinguish between different layers in the epidural space and thus indicate the position of the needle.

Optionally the optical source and optical detector are arranged for diffuse reflectance measurements, the implementation of which is now described. Other optical methods are also applicable for the extraction of tissue properties such as diffuse optical tomography by employing a plurality of optical fibers, differential path length spectroscopy, Fluorescence and Raman spectroscopy. A good discussion on diffuse reflectance measurements is given in R. Nachabé, B. H. W. Hendriks, A. E. Desjardins, M. van der Voort, M. B. van der Mark, and H. J. C. M. Sterenborg, “Estimation of lipid and water concentrations in scattering media with diffuse optical spectroscopy from 900 to 1600 nm”, J. Biomed. Opt. 15, 037015 (2010). In this, either the optical radiation source or the optical detector or a combination of both are arranged to provide wavelength selectivity. For instance, light can be coupled out of the distal end of at least one optical waveguide, which serves as a source optical waveguide, and the wavelength is scanned, for example from 0.5 μm to 1.6 μm, while the optical radiation detected by the at least one optical waveguide in communication with an optical detector, is sensed by a broadband optical detector. Alternatively, broadband radiation can be provided by at least one source optical waveguide, while the optical radiation collected by at least one optical waveguide in communication with an optical detector is sensed by a wavelength-selective optical detector, for example a spectrometer.

Optionally the collected optical signal is further processed using an algorithm in order to derive the optical properties of tissue in contact with the distal end of the waveguide. These include the scattering coefficient and absorption coefficient of different tissue chromophores such as hemoglobin, oxygenated hemoglobin, water, and fat. Since these properties vary between the different layers in the spinal column shown in FIG. 1 the collected optical signal can be used to distinguish between the Epidural Space, nerves and blood vessels and surrounding tissues.

The algorithm described in more detail as follows. The spectral fitting is performed by making use of the analytically derived formula for reflectance spectroscopy as described in R. Nachabé, B. H. W. Hendriks, A. E. Desjardins, M. van der Voort, M. B. van der Mark, and H. J. C. M. Sterenborg, “Estimation of lipid and water concentrations in scattering media with diffuse optical spectroscopy from 900 to 1600 nm”, J. Biomed. Opt. 15, 037015 (2010) and in T. J. Farrel, M. S. Patterson and B. C. Wilson, “A diffusion theory model of spatially resolved, steady-state diffuse reflectance for the non-invasive determination of tissue optical properties,” Med. Phys. 19 (1992) p 879-888.

This reflectance distribution R is given by:

$$\begin{matrix}R ( ρ ) = {\int }_{0}^{∞} R ( ρ , {z}_{0} ) δ ( {z}_{0} - 1 / {μ}_{t}^{′} ) d {z}_{0} \\ = \frac{{a}^{′}}{4 π} [ \begin{matrix}\frac{1}{{μ}_{t}^{′}} ( {μ}_{eff} + \frac{1}{{r}_{1}} ) \frac{{e}^{- {μ}_{eff} {r}_{1}}}{{r}_{1}^{2}} + \\ ( \frac{1}{{μ}_{t}^{′}} + 2 {z}_{b} ) ( {μ}_{eff} + \frac{1}{{r}_{2}} ) \frac{{e}^{- {μ}_{eff} {r}_{2}}}{{r}_{2}^{2}}\end{matrix} ]\end{matrix} \\ where \\ {r}_{1} = {[ {x}^{2} + {y}^{2} + {( 1 / {μ}_{t}^{′} )}^{2} ]}^{1 / 2} \\ {r}_{2} = {[ {x}^{2} + {y}^{2} + {( ( 1 / {μ}_{t}^{′} ) + 2 {z}_{b} )}^{2} ]}^{1 / 2} \\ {μ}_{eff} = \sqrt{3 {μ}_{a} [ {μ}_{a} + {μ}_{s} ( 1 - g ) ]} \qquad ( 1 )$$

In this formula the three macroscopic parameters describing the probability of interaction with tissue are: the absorption coefficient μₐ and the scattering coefficient μₛ both in cm⁻¹ as well as by g which is the mean cosine of the scattering angle. Furthermore, the total reduced attenuation coefficient μₜ′ is used which gives the total chance for interaction with tissue:

μ′=μₐ+μₛ(1−g). (2)

The albedo a′ is the probability of scattering relative to the total probability of interaction

a′=μₛ/μₜ′. (3)

A point source at a depth z₀=1/μₜ′ is assumed, together with no boundary mismatch hence z_{b}=2/(3μₜ′). Furthermore, it is assumed that the scattering coefficient can be written as

μₛ′(λ)=aλ^{-b}. (4)

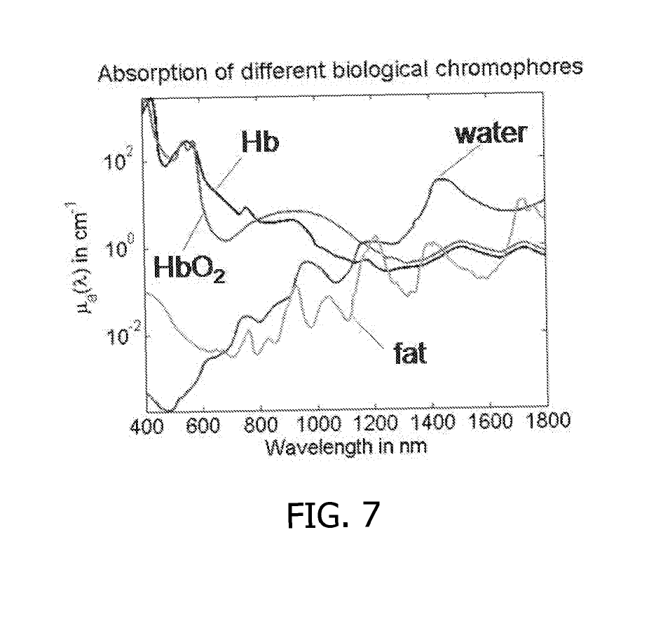

The main absorbing constituents in normal tissue dominating the absorption in the visible and near-infrared range are blood (i.e. hemoglobin), water and fat. FIG. 7 shows graphically the absorption of different biological chromophores as a function of optical wavelength. In this it is noted that blood dominates the absorption in the visible range, while water and fat dominate in the near infrared range.

The total absorption coefficient is a linear combination of the absorption coefficients of for instance blood, water and fat. By fitting the above formula while using the power law for scattering, the volume fractions of the blood, water and fat are determined as well as the scattering coefficient. With this method the measured spectra are translated into physiological parameters that can be used to discriminate different tissues.

Alternatively, principal components analysis can be used as a means of discriminating tissue. This method allows classification of differences in spectra and thus allows discrimination between tissues. Alternatively, it is also possible to extract features from the spectra as discussed in WO2011132128.

Optionally the optical detector may be further configured to measure a selection of optical parameters for the optical source by means of an optical beamsplitter in order to compute changes between the optical source radiation and that collected at the distal end of the waveguide. A beamsplitter is an optical component that when placed in the optical path acts to redirect a portion of the incident optical radiation whilst simultaneously permitting the transmission of the remaining portion of the incident optical radiation. One example implementation comprises a mirror having 50% reflectance and 50% transmission which is placed at 45 degrees to the incident beam. Thus such a beamsplitter placed between the optical source and the source optical waveguide at 45 degrees to the beam of the incident optical radiation may be used to redirect a portion of the source optical radiation toward an optical detector in order to measure a property of the source optical radiation. At the same time the remaining portion of the source incident radiation is permitted to pass through the beamsplitter and consequently along the source optical waveguide to irradiate tissue at the distal end of the elongate tube. According to this example the radiation collected by the detector optical waveguide at the distal end of the elongate tube may be directed to the same, or to a further optical detector to that measuring the source optical radiation. When two optical detectors are used, a first optical detector may thus be configured to generate a response to only the optical source radiation, and a second optical detector may thus be configured to generate a response to only the radiation collected by the detector optical waveguide. The ratio of the response generated by the second optical detector to the response generated by the first optical detector may thus be used to correct for variations in source optical power. When a single optical detector is used the radiation from the optical source and the optical radiation collected by the detector optical waveguide are both directed to the same detector and thus the detector may be used to generate a response to the sum of the two sources of radiation. By providing an additional optical shutter which is configured to temporally interrupt the radiation from either the detector optical waveguide or the source from reaching the detector, the shutter may be used to arrange that the detector generates either a response to the optical source radiation, to the radiation collected by the detector optical waveguide, or to both the optical source radiation and the radiation collected by the detector optical waveguide. By appropriately differencing and taking the ratio of the generated signals the single optical detector may be used to correct for spurious variations in both the source optical power and in the detector's responsivity.

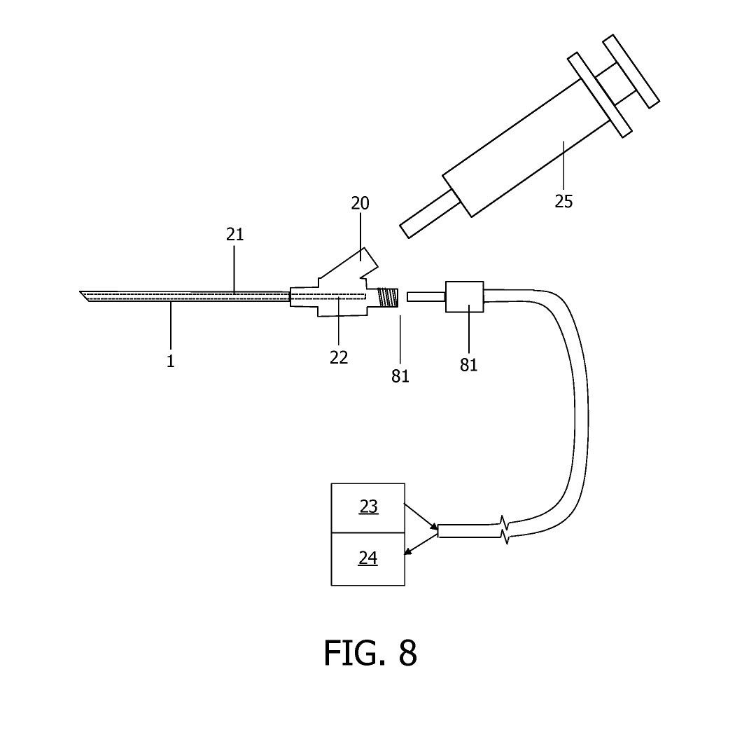

Optionally the medical needle is further provided with at least one optical connector at the proximal end of the elongate tube. FIG. 8 shows schematically an example arrangement of the invention being further provided with an optical connector at the proximal end of a needle. In FIG. 8, the mating components of an SMA-style optical connector with a screw thread 81 is used to facilitate the communication with the optical source and the optical detector. In this example a single optical connector is shown for use in the situation in which the functionality of the optical waveguides are combined into a single waveguide, for example in the use of Raman spectroscopy in which a single waveguide is sometimes used. Alternatively the medical needle may be further provided with more than one optical connector in for example the situation where it is desirable to use a separate optical waveguide in communication with the optical source to that in communication with the optical detector.

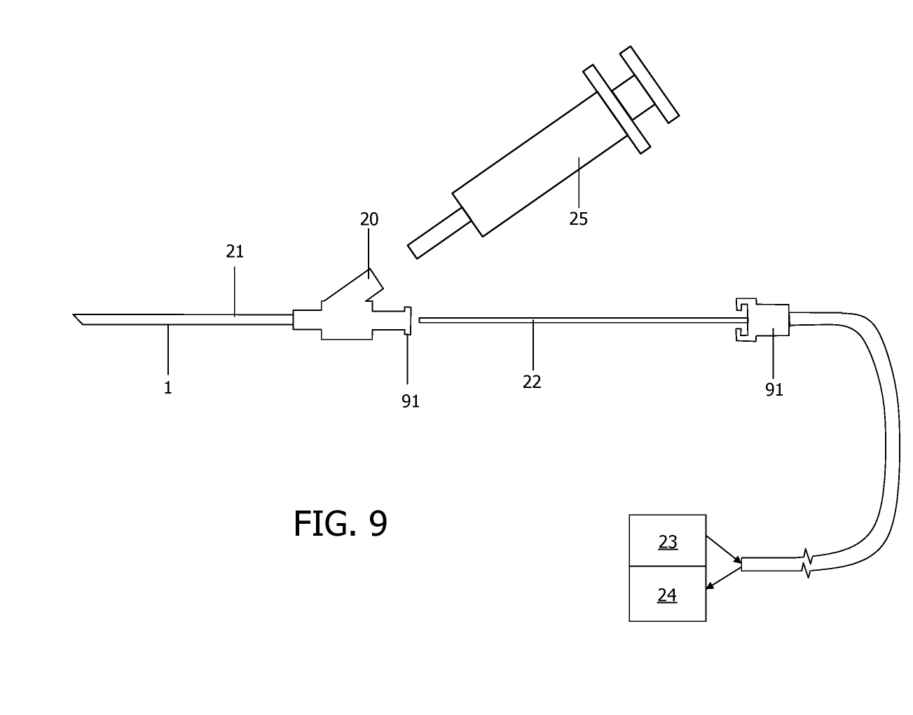

Optionally the medical needle is further provided with at least one mechanical fastening at the proximal end of the needle. FIG. 9 shows schematically an example arrangement of the invention being further provided with a mechanical fastening at the proximal end of a needle. In FIG. 9 the mating components 91 of a snap connector are used to fix the one or more optical waveguides with respect to the proximal end of the needle when the optical waveguides 22 are inserted into the needle 1. In so doing the one or more mechanical fastenings permit the temporary insertion of the one or more optical waveguides into the needle during use, allowing for the later disposal of the needle.

To summaries, a medical needle has been described based on an example of epidural anesthesia, which comprises an elongate tube having a distal end and a proximal end, a syringe connector, at least one channel and at least one optical waveguide. The cross section of the distal end of the elongate tube has a dividing line for each channel which is tangential to the cross section of that channel and transverse to the tube's longitudinal axis. Furthermore, the distal end of that channel is arranged to lie on one side of said dividing line and the distal end of the one or more optical waveguides are arranged to lie on the opposite side of said dividing line.

While the invention has been illustrated and described in detail in the drawings and foregoing description, such illustrations and description are to be considered illustrative or exemplary and not restrictive; the invention is not limited to the disclosed embodiments and can be used for various types of medical probes.

1. A medical needle comprising
an elongate tube having a distal end and a proximal end and a single bore,
a syringe connector,
at least one channel,
two or more optical waveguides,
a stylet insert having at least one lumen,
wherein
the elongate tube is open at the distal end,
the at least one channel is formed within the tube,
the syringe connector is in communication with the at least one channel, the distal ends of the two or more optical waveguides are fixed with respect to the long axis of the elongate tube,
the two or more optical waveguides are arranged within the at least one lumen of the stylet insert, and the stylet insert is further inserted into the single bore of the elongate tube,
the distal end of the cross section of stylet insert is shaped such that it does not completely fill the bore into which it is inserted and the at least one channel is formed by leaving part of the cross section of the bore free from the stylet insert,
the cross section of the distal end of the elongate tube has a dividing line for each channel which is tangential to the cross section of that channel and transverse to the tube's longitudinal axis and the distal end of that channel is arranged to lie on one side of said dividing line and the distal ends of the two or more optical waveguides are arranged to lie on the opposite side of said dividing line.

2. A device according to claim 1 wherein at least a portion of the outer cross section of the stylet insert fits to the inner cross section of the bore into which it is inserted, such that for this portion the outer surface of the stylet insert is in intimate contact with the inner cross section of the bore into which it is inserted.

3. A device according to claim 2 wherein the distal end of the elongate tube has a bevel and the distal end of the stylet insert has a bevel with substantially the same bevel angle, the stylet insert being further arranged within the elongate tube such that the bevel of the stylet insert and the bevel of the elongate tube are substantially coincident.

4. A device according to claim 3 having at least one optical waveguide in communication with an optical source, namely at least one source optical waveguide, and having at least one optical waveguide in communication with an optical detector, namely at least one detector optical waveguide, wherein the at least one source optical waveguide is separate to the at least one detector optical waveguide.

5. A device according to claim 4 wherein the end face of the beveled distal end of the elongate tube has a second dividing line that is parallel to the short axis of the bevel, wherein the distal end of the one or more source optical waveguides are arranged to lie on a first side of said second dividing line and the distal end of the one or more detector optical waveguides are arranged to lie on a second side of said second dividing line.

6. A device according to claim 5 wherein the two or more optical waveguides comprise at least one optical fiber.

7. A device according to claim 1 being further provided with a look-up table comprising the optical properties of human tissue at optical wavelengths and an optical detector, wherein the optical detector is arranged for the generation of an optical response to radiation collected at the distal end of the elongate tube, and in which the tissue in contact with the distal end of the elongate tube is determined from the optical response and the look-up table.

8. A needle positioning arrangement comprising the device of claim 1 being further provided with a syringe and an Acoustic Pressure Assist Device for applying pressure to the syringe and providing continuous acoustic feedback relating to the pressure exerted by the syringe at the distal end of the elongate tube, wherein the syringe is connected to the syringe connector and the syringe is in communication with the Acoustic Pressure Assist Device.
